(19) United States
(12) Patent Application Publication
(10) Pub. No.: US 2008/0069941 A1
(43) Pub. Date: Mar. 20, 2008

(54) Animals for Conserving N-3 Highly Unsaturated Fatty Acids

(75) Inventor: Karl Thomas Geiringer, Waitakere City (NZ)

Correspondence Address: YOUNG & THOMPSON, 745 SOUTH 23RD STREET, 2ND FLOOR, ARLINGTON, VA, 22202, US

(73) Assignee: PHOTONZ CORPORATION LIMITED, WAITAKERE CITY (NZ)

(21) Appl. No.: 11/662,413

(22) PCT Filed: Sep. 13, 2005
(86) PCT No.: PCT/NZ05/00244

§ 371 Date: Aug. 7, 2007

(30) Foreign Application Priority Data
Sep. 13, 2004 (NZ) 535321

(51) Int. Cl. A23C 9/00; A01K 67/00; A23D 7/00; C12N 15/63; C12N 5/00

(52) U.S. Cl. 426/580; 426/608; 435/320.1; 435/325; 800/8

(57) ABSTRACT

Food-providing animals for providing human food rich in at least one of the health-promoting n-3 HUFAs (omega-3 highly unsaturated fatty acids) are selected and then bred so that a greater proportion of dietary n-3 HUFAs and/or their metabolic precursors provided to the animals is conserved in the animals food tissue as n-3 HUFA and transferred into human food. Evidence of heritability, methods for phenotypic or genotypic identification and selection of animals genetically adapted for that function are provided. Animals include farmed mammals and birds, and marine or fresh water animals including fish, crustaceans and molluscs.

### FIELD

This invention relates to the identification and provision of food-providing animals having an enhanced and heritable ability to conserve N-3 highly unsaturated fatty acids.

### DEFINITIONS AND ABBREVIATIONS

Fatty acid refers to a chemical compound having a backbone of carbon atoms, the bonds between some of which may be unsaturated, with an acid (COOH) moiety at an alpha end in the case of the free acid. The other end is the omega (ω) end. For the purpose of this specification, the term does not exclude salts and esters (including but not limited to ethyl or cholesterol esters, amides, phospholipids, or mono, di- or tri-glycerides) thereof.

HUFA is used herein as an abbreviation for a highly unsaturated fatty acid having five or more unsaturated double bonds between carbon atoms.

n-3 HUFA refers to the group of omega-3 HUFAs having at least five double bonds, the first of which is located three carbon residues from the omega end of the molecule EPA, DPA and DHA, are members of the n-3 HUFA group.

C20:5 and EPA are abbreviations used herein for eicosapentaenoic acid (icosapentaenoic acid).

C22:5 and DPA are abbreviations used in this text for docosapentaenoic acid.

C22:6 and DHA are abbreviations used in this text for docosahexaenoic acid.

n-3 HUFA tissue refers to tissues (and secretions therefrom) having a modified fat composition principally in relation to the proportion of n-3 HUFAs therein.

Micro-algae refers to single-celled microscopic plant-like organisms such as phytoplankton or diatoms (as distinct from polycellular algae such as seaweeds).

Animal refers in this context to food-producing creatures and to progenitors thereof, such as, without limit, dairy cows, beef animals, other farmed mammals including sheep, water buffalo, and goats; poultry or farmed fish, reptiles, crustacea and molluscs all of which may be identified, selected and bred. Although the Examples herein tend to focus on dairy cows the same principles are generally applicable throughout.

Conservation describes a property held by an animal of holding n-3 HUFA in its tissue. This property may be related to the ability of the animal to retain an ingested molecule or part thereof without conversion into another molecule by a substantially irreversible metabolic process. Conservation allows recovery of substantial amounts of desired molecules or parts thereof in a food obtained from the animal. (By referring to “part” this definition embraces ingestion of metabolic precursors of molecules and their conversion into desired molecules that retain substantial parts of the original molecules).

Tissue or secretions as used herein refers to edible products obtainable from food-producing animals wherein “tissue” includes meats, offals, blood and fat as well as the entire corpus (in the case of molluscs for example); and “secretions” includes milk and bird's eggs.

Edible products as used herein also includes processed derivatives of animal products, such as milk powder, butter, and cheese from milk; pate, meat extracts, egg extracts and the like.

### BACKGROUND

Preventative health recommendations (WHO 2001; World Health Organization, Avenue Appia 20, 1211 Geneva 27, Switzerland) exist to increase the omega-3 highly unsaturated fatty acid (n-3 HUFA) component of the human diet. Such an increase should improve at least cardiovascular function; also brain development and function, utilise anti-inflammatory properties of n-3 HUFAs, and possibly reduce sequelae of obesity. n-3 HUFAs such as EPA, DPA and DHA, as components of food and or active constituents of supplements, are also widely recognised as having important therapeutic benefits in human health and in medicine. The level of n-3 HUFA in animal tissue consumed by humans has generally decreased due to changes in the diet supplied to animals and the way such foods are processed. It may also be that to some extent that existing breed selection programmes select for traits that dilute the ability of farmed animals to conserve these fatty acids in tissue. It would be generally useful to raise the amount of n-3 HUFAs ingested by humans, which infrequently reaches the recommended targets (for EPA and DHA) of about 250-500 mg per person per day. That dietary target is hard to satisfy for a population given the limitation in accessibility and acceptability of appetising products derived directly from marine algae, and the low global intake of fish as a form of animal protein, exacerbated by dwindling fish stocks. A metabolic precursor, alpha linolenic acid (ALA), which can be used by humans to a limited extent in the synthesis of n-3 HUFAs, is also deficient in the diets of most people in affluent societies for reasons such as processing damage, shelf life considerations, and changes in food choices. For the reasons stated above the effective intake of n-3 HUFAs or their precursors has decreased to less than 20% of what was present in common diets 150 years ago. Additionally, the ratio of omega-3 (n-3) to omega-6 (n-6) fatty acids in the human diet has also decreased significantly in recent years. A low n-3 to n-6 ratio is believed to be adverse at typically about 1:15 whereas high ratios of about 1:2 to 1:4 have been associated with reduced mortality from cardiovascular disease and other diseases. Provided the total amount of n-6 HUFA consumed by humans does not rise simultaneously, it may be possible to improve this ratio by raising the total amount n-3 (including n-3 HUFA) consumed. Even if the n-6 component of the human diet does not decrease, a significant increase in consumption of n-3, and in particular n-3 HUFAs over today's levels should benefit human health. It is known that n-3 FAs tend to some extent to be conserved within an organism from ingestion to eventual consumption by other organisms. In this way n-3 HUFA may be retained along several links of a food chain (for example alga—crustacean—fish—predatory fish). One way to achieve an increase in n-3 HUFA consumption by humans therefore would be to raise significantly the n-3 HUFA component of human foodstuffs made from food-producing animals. In addition it is known that to some extent precursor n-3 fatty acids such as ALA, which can be found in flax seeds and canola, and also to a small extent stearidonic acid (SDA) found in some other seeds including certain hemp varieties, can be metabolised by animals and conserved in tissue as n-3 HUFAs. There have been trials in which n-3 HUFAs and/or their metabolic precursors were fed to food-producing animals and conservation of the n-3 HUFAs was shown. For example cows fed on fish extracts rich in n-3 HUFAs and/or their precursors have produced butterfat having a raised proportion of n-3 HUFAS. N-3 HUFAs and/or their precursors provided in the diet of food producing animals is incorporated in the food tissues of such animals at various rates as a result of processes related to the extent to which such fatty acids are (a) able to be metabolically converted into n-3 HUFAs by the animal concerned, (b) sequestered into food versus non-food tissues, (c) oxidised as part of the animals' energy metabolism or to maintain fatty acid homeostasis, (d) irreversibly metabolised to support growth, continual maintenance and repair, (e) secreted as fats such as those found in milk. Although these processes are known to vary (in some cases markedly) between individuals, prior to the current invention it had not been observed that these processes can vary in a predictable way according measurable genetic characters. Nor had it been shown how these characters could be used to produce food for human consumption containing significantly increased levels of n-3 HUFA.

### PRIOR ART

Cooper, in US 2003/0039737 describes acquiring a population of cows that produce a desired fat composition in their milk by testing a number of cows fed with ordinary pasture, segregating those that have a suitable fat composition, and breeding from those cows. 5 to 10% of New Zealand Friesian breed cows inherently produce a suitable fat composition. Suitable cows would be located by tests of their milk fat composition “when in standard farm conditions” and suitable sires for use over these cows would (a) have high genetic merit and (b) be known to generate many daughters with a suitable fat composition. Cooper refers to “modified feeds” including one or other of (a) processing to cause protection of a food from microbial consumption within the rumen yet allow later absorbtion and (b) including supplements of unsaturated fatty acids and their metabolic precursors, however the methods provided by Cooper for obtaining low saturate, high monounsaturate (MUFA milk) may in fact teach away from the production of high n-3 HUFA animal tissues. (Franklin et al (J Nutr (1999) 129(11) 2048-2053) had tested protection of foodstuffs of micro-algal origin against ruminal micro-organisms by encapsulation). Cooper's method is concerned with production of milk with low saturated fatty acid and high monounsaturated fatty acid levels and whilst it is true that under certain conditions the methods disclosed by Cooper may lead to milk with relatively high ALA and even increased polyunsaturated fatty acid levels overall they favour a reduced n-3 to n-6 ratio which may quite apart from being suboptimal for human health, inhibit subsequent metabolic conversion of ALA to n-3 HUFA leading to lower milkfat n-3 HUFA levels. Additionally neither Cooper nor previous commentators provide methods of selecting animals for their ability to conserve n-3 HUFAs by supplementing the diet of animals prior to phenotypic or genotypic selection.

### OBJECT

It is an object of this invention to provide methods for identifying food-producing animals capable of conserving dietary n-3 HUFAs in proportions beneficial to human health, and to provide a population of the animals themselves, having a heritable and improved ability to produce foods having a modified fat composition beneficial to human health, or at least to provide the public with a useful choice.

### STATEMENT OF INVENTION

In a first broad aspect this invention provides a food-producing animal selected from the range of food-producing animals as herein defined, wherein the animal has a heritable capability for conservation (as herein defined) of at least one compound selected from a range including: the n-3 HUFAs: EPA (eicosapentaenoic acid), DPA (docosapentaenoic acid) and DHA (docosahexaenoic acid) and/or metabolic precursors thereof, so that the animal is capable of producing a food product including a high level of at least one of the n-3 HUFAs as compared to levels of n-3 HUFAs in foods derived from animals lacking said heritable capability.

In a first related aspect this invention provides a food-producing animal selected from the range of food-producing animals as herein defined, wherein when fed with a diet including a supplementary amount of at least one compound selected from a range including: the n-3 HUFAs: EPA (eicosapentaenoic acid), DPA (docosapentaenoic acid), and DHA (docosahexaenoic acid) and/or metabolic precursors thereof, the food-producing animal demonstrates a heritable capability of conserving at least one n-3 HUFA at an effective level in food tissue so that the animal is capable of producing a food product including a higher level of at least one of the n-3 HUFAs as compared to levels in foods derived from animals either lacking said heritable capability or not provided with the augmented diet.

In a second related aspect this invention provides a breeding population of food-producing animals as previously described in this section, wherein the population of food-producing animals exists, whether physically brought together or aggregated in an informational manner, in a sufficient number to be capable of transmitting the heritable capability to progeny of the population without adverse genotypes arising in the progeny owing to inbreeding, and wherein the number is in the range of from at least 10 to at least 50 breeding animals.

In a second broad aspect the invention provides a method for obtaining members of a population of food-producing animals as as previously described in this section, wherein the method involves the steps of: obtaining access to a base population of individually identified food-producing animals, each animal having at least one identified parent, carrying out a first test procedure on members of the base population; the first test procedure including the steps of (i) supplementing the diet of each animal to be tested with a supplement rich in at least one n-3 HUFA or a precursor thereof at an effective level for a period, (ii) collection of an appropriate tissue sample from the animal, (iii) collecting and analysing the fatty acid composition of the sample, then (iv) recording the amount and percentage of total fatty acid comprised of at least one n-3 HUFA, and identifying and if necessary separating out the animals providing the highest test results as comprising the selected population of animals.

In a first related aspect this invention provides a method for obtaining a food-producing animal, wherein the method involves the steps of: obtaining access to a base population of individually identified animals, each animal having an identified sire selected from within a limited group of sires, carrying out the first test procedure on members of the base population, ascertaining the ranking of the sires of the members within the group of sires in terms of measurements derived from the first test procedure of samples taken from their progeny and ranking those sires whose progeny tends to return the highest amounts of n-3 HUFAs in the first test procedure relative to unrelated companions as the highest ranked sires, and identifying and if necessary separating out the animals that are the progeny of the highest ranked sires as comprising the selected population of animals.

In a second related aspect this invention provides a food-producing animal, wherein the animals that are (a) the progeny of the highest ranked sires and (b) are themselves highly ranked in terms of n-3 HUFA measurements as determined by the first test procedure are defined as comprising the selected group of animals.

In a third related aspect this invention provides a food-producing animal, wherein the method uses an alternative, second test procedure that does not include the step in the first test procedure of supplementing the diet of each animal to be tested with a supplement rich in at least one n-3 HUFA or a precursor thereof at an effective level for a period before taking the sample.

In a fourth related aspect this invention provides a food-producing animal, wherein the method uses an alternative, third test procedure instead of the first or the second test procedures namely: carrying out a third test procedure on members of the base population; the third test procedure including the steps of (i) collecting a sample of genetic material from the animal under test and analysing the genetic composition of the sample, then (ii) recording the genetic composition of the animal in terms of at least one of: genes relevant to conservation of the n-3 HUFAs, genetic markers associated with genes relevant to conservation of the n-3 HUFAs, and expressed m-RNA or expressed proteins indicative of genes relevant to conservation of the n-3 HUFAs.

In a third broad aspect the invention provides a method for maintaining a selected group of food-producing animals wherein the method includes the steps, taken for each generation, of (a) repeating the procedure of ascertaining the performance of the food-producing animals within the selected population in terms of n-3 HUFA measurements from their progeny according to the first test method, (b) ranking those food-producing animals whose progeny return the highest proportions of n-3 HUFA fats in the tests as the highest ranked breeding animals, and (c) breeding from the highest ranked breeding animals.

Preferably the extent of inbreeding that may occur is minimised by the step of endeavouring to breed between animals that are as distantly related as possible, so that the population does not develop a high frequency of recessive genes and so that the effective breeding number of the population is maximised.

Preferably the majority of individual animals in the population will express significantly higher n-3 HUFA in selected tissues or secretions than a population of the same animals maintained through conventional breeding techniques and fed the same diet, the method comprising (A) sampling selected animal tissue (B) investigating the sample to ascertain the proportion of n-3 HUFAs in the tissue, (C) repeating steps A and B with additional individual animals until (D) a number of animals have been shown to produce n-3 HUFA rich tissue, and (E) assigning unique identifiers to those animals, and (F) managing those animals for the purpose of producing food products as a population distinct from animals determined not to produce n-3 HUFA-rich tissue or secretions.

In a first related aspect the invention provides reproductive material carrying genetic material capable of contributing to the genomes of a food-producing animal as previously described in this section, wherein the reproductive material has been derived from a breeding method as previously described in this section; the reproductive material being selected from a range including spawn, milt, eggs and semen; embryos, larvae, stem cells, and intermediate and final products of genetic engineering including genetic constructs.

In a fourth broad aspect the invention provides an optionally processed foodstuff of animal origin, wherein the foodstuff is obtained from at least one animal obtained by a process as previously described in this section and includes a raised proportion of at least one compound selected from the range including: the n-3 HUFAs: EPA (eicosapentaenoic acid), DPA (docosapentaenoic acid) and DHA (docosahexaenoic acid).

In a first related aspect the invention provides a dairy foodstuff as previously described in this section, wherein the foodstuff is derived from secreted milk from at least one dairy animal, so that the dairy foodstuff has a raised proportion of n-3 HUFAs, and preferably pooled with milks from like animals.

In a related aspect the invention provides at least one manufactured product made from a pooled product as previously described in this section; the products including without limitation dairy products such as powdered milk, condensed milk, skim milk, cream, butter, cheese, chocolate, ice cream, yoghurt, infant formulations, and forms of milk having altered protein compositions.

A foodstuff of avian origin as previously described in this section, wherein the foodstuff comprises at least one egg or derivatives thereof.

A foodstuff as previously described in this section, wherein the foodstuff comprises a body tissue selected from the range including meat or offal or blood.

In a related aspect the invention provides at least one manufactured product made from a pooled product as previously described in this section; the products including without limitation meat or other tissue-based products such as sausage meat, mince, processed chicken, and other packages or compositions including parts from more than one individual animal.

### PREFERRED EMBODIMENT

The description of the invention to be provided herein is given purely by way of example and is not to be taken in any way as limiting the scope or extent of the invention.

### LIST OF ILLUSTRATIONS

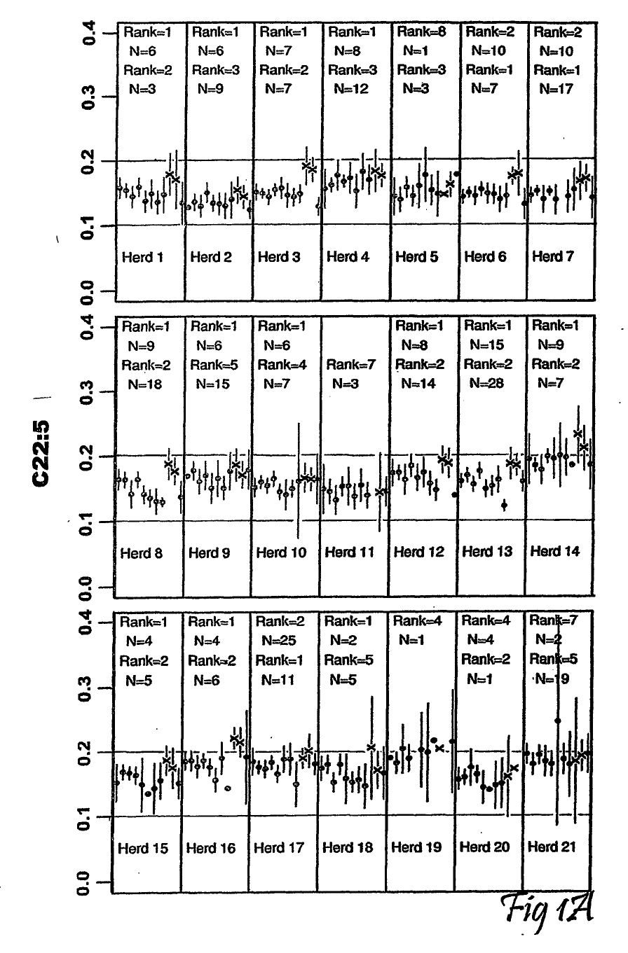

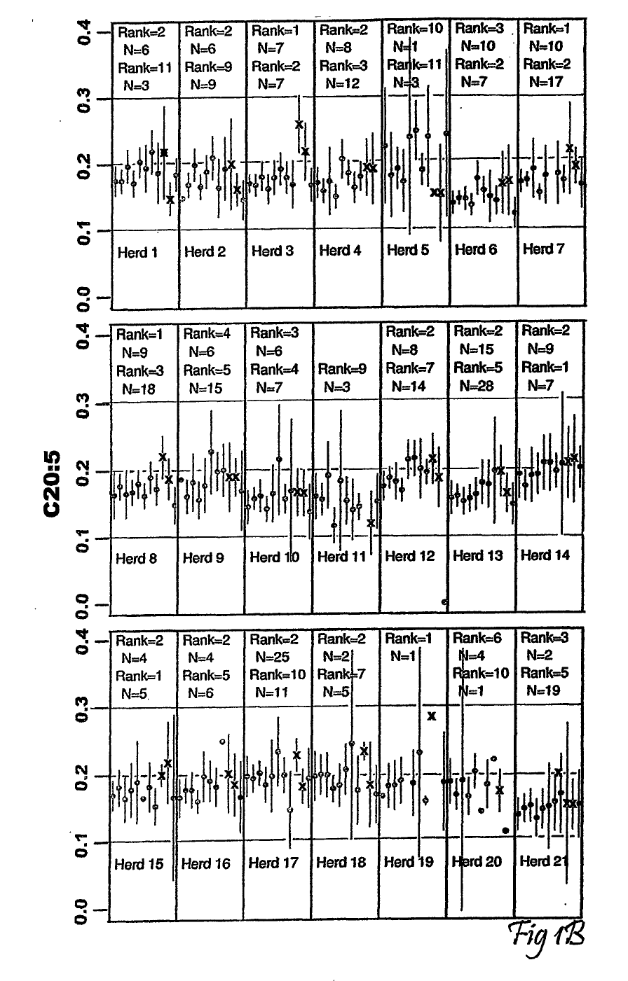

FIG. 1: as two sheets, FIG. 1a (C22:5) and FIG. 1b (C20:5) shows a statistical ranking of daughters of sires in a breeding experiment carried out in data obtained from New Zealand Friesian cows.

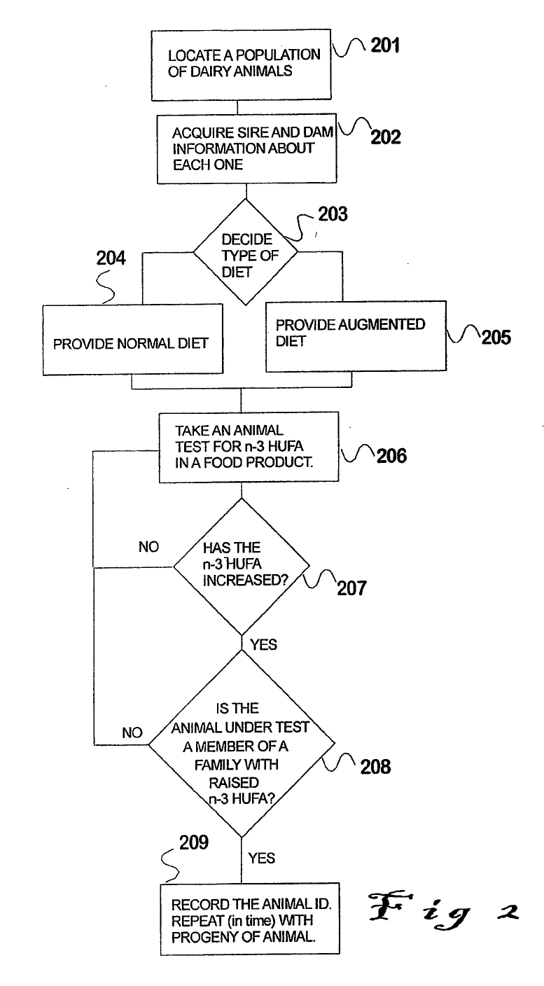

FIG. 2: shows an example flow chart for selecting food-producing animals according to the invention.

The invention intends to maximise conservation of n-3 HUFAs by new and existing strains of known food-producing animals until the compounds, in particular EPA, DPA and DHA, appear in the foods so produced at usefully raised levels by a process of selection of heritable characters on a phenotypic or a genotypic basis. Such heritable characters may also maintain conservation if fed a diet or dietary supplement including the n-3 HUFAs (or metabolic precursors such as alpha-linoleic acid). As a result foods made from the animals or their secretions comprise a more healthy version of foodstuff of animal origin for human consumption. The health of the human population is thereby improved. A breeding programme intended to build up herds of animals that are inherently capable of producing animal-based food products having useful amounts of n-3 HUFAs is proposed. At this point the upper limits in terms of cost-effective concentration of preferred HUFAs within acceptable foods has not been determined.

The invention is particularly directed to identifying and breeding those animals whose genotypes have the effect of conserving n-3 HUFA levels, when fed a supplemented diet in addition to identification of those animals that have relatively higher n-3 HUFA levels after being fed an ordinary diet including no supplements. The exact mechanism of this phenotypic characteristic is as yet unresolved although it is most likely to be mediated through relative enzyme activity along the relevant and well-known biochemical pathways in the animals' bodies. There are likely to be a number of genes involved. Further, it is not impossible that the higher n-3 HUFA measured on analysis occurs as a result of reduced expression of particular genes, such as genes responsible for biochemical pathways converting fatty acids into energy, in combination with hyper-activity or raised expression of the products of other genes.

Genes of interest include delta-6 desaturase, delta-5 desaturase, and the set of fatty-acid chain elongation enzymes, and genes that affect the expression or activity of the listed genes. Other genes of interest include those that express or modulate gut-wall absorbtion proteins, transport and binding proteins acting on fatty acids, particularly any acting on n-3 HUFAs as found within the gut lining, also plasma, cell membranes and other tissues (including mammary tissues). Further genes of interest include those that lead to loss of n-3 HUFAs through unwanted metabolic conversion or peroxidation. Genetic markers, polymorphism in relevant alleles, m-RNA and expressed proteins are also of interest.

### EXAMPLE 1

### Dairy Cows

The experiment consisted of evaluating the milk fat composition of 4100 New Zealand Friesian cows, the progeny of unspecified dams and of 11 identified artificial breeding sires in 21 herds located within the provinces of Northland, Waikato and Taranaki in New Zealand; run under standard commercial conditions and fed a conventional diet. The diet of each herd would include a variable amount of unsaturated fatty acids and precursors thereof, and a statistical smoothing (by ranking) of results is useful in order to show trends. Of the 11 sires, two (sires A and B) which are half-brothers having a common sire were found when breeding records were also inspected to have sired progeny having interesting results as shown in Table 1 and FIGS. 1A and 1B in terms of rank. Test data was obtained some time ago. The statistical technique is described below.

TABLE 1

| | C22:5 DPA | | C20:5 EPA | |
|---|---|---|---|---|
| 21 herds. | rank sum | | rank sum | |
| Sire A | 32 | p < 0.001 | 43 | p < 0.001 |
| Sire B | 42 | p < 0.001 | 101.5 | p < 0.32 |
| Possible range of | 11-225 | | 11-225 | |
| rank | | | | |

This Example is partly based on the discovery that particular dairy sires are capable of producing progeny having milk fats that, when tested under commercial pasture-fed conditions, have a significantly higher mean concentration of DPA and EPA than the mean of milk fats of progeny of other dairy sires. The higher concentration becomes more evident when the performance of the peers is ranked within herds. (See FIGS. 1A and 1B, and Table 1). In these Figures, up to 11 milk-fat test results from milk taken from daughters of each sire within each herd are shown separately in left to right order in a corresponding box (one of three rows of seven) labelled Herd 1 to 21. The percentage in total milk fats of the compound of interest is shown on the scale at left. The two sires (A and B) that showed this useful property in their progeny (each marked “X” were themselves half-sibs. Progeny of other sires are marked with dots, and all have variance bars. We believe that maintaining the herd data separately for analysis was useful in order to overcome other types of variations between farms.

Please note that the experiment giving rise to this particular set of data did not include the optional but preferred step of feeding the animals under test with supplementary diets rich in the n-3 HUFAs and/or their precursors. We do not have any evidence that supplementation will result in an increase of n-3 HUFAs in the milk of the same superior animals as were identified in this experiment. The ability of an animal to conserve n-3 HUFAs from a supplement given as part of the diet may or may not be related to the ability of an animal to conserve n-3 HUFAs when fed a conventional non-supplemented diet. One logical next step would be to isolate the progeny of at least sires A and B and measure their ability relative to a random and statistically significant selection of unrelated counterparts to conserve n-3 HUFA when supplemented at a range of supplemented input levels of n-3 HUFAs and/or their precursors exceeding the levels found in pasture.

The outcome of this trial in terms of the raised levels of n-3 HUFAs achieved in food tissue is clearly insufficient in itself to satisfy an average daily requirement for a person consuming an average amount of dairy fat. An about 30 percent increase in EPA in butterfat is demonstrated. However, even where this may have no directly measurable effect on the health of an individual person, the health of a population consuming, as a whole, the incremental increase such an individual might accrue, should show an improvement especially if any confusing effects are eliminated. Manufactured products made from a pooled raw product from a population of animals according to this invention include, without limitation, products such as meat or other tissue-based products such as sausage meat, mince, processed chicken, and other packages or compositions including parts from more than one individual animal.

Some Proposed Actions:

1. A breeding programme intended to ascertain whether further generations of selection would result in higher rankings or other improvements in DPA and EPA (or possibly even DHA or and/or other n-3 HUFA) concentrations has not yet been done. That is a time-consuming process. It may be advisable to first locate another sire that is not related to sires A and B yet has similar attributes in respect of DPA and EPA in milk fat of progeny, and use this sire over the identified higher ranked cows sired by sires A or B so that adverse results of inbreeding are less likely to occur. One desire in this invention is of course to produce a stock of animals that will “breed true” for this attribute over a number of generations and the deleterious (often recessive) gene frequencies seen on inbreeding would be unhelpful.

We expect that animals so selected will be more efficient at supplying animal-based foods including desired amounts of the HUFAs than unselected animals. The effect may be accentuated if fed on high-HUFA raw materials (such as foods based on or including algal materials, or some fish or other marine foods and including the n-3 HUFA precursors alpha-linoleic acid, linolenic acid, and the like, as found in flax seed oil, canola oil, and in other plant sources.

2. A preferred protocol for testing, as shown in the flow chart of FIG. 2, includes the steps of: obtaining access to a base population of individually identified food-producing animals, each animal having at least one identified parent selected from within a limited group of parents, then carrying out a first test procedure on members of the base population. A preferred test procedure includes the steps of supplementing the diet of each animal to be tested with a supplement rich in at least one n-3 HUFA or a precursor thereof at an effective level for a period prior to collection of an appropriate sample from the animal. The scoring process should take notice of any evidence of heritability. This is a matter of picking up as much pedigree data as possible from the measurements of n-3 HUFAs as well as from sire and dam information which may go back several generations, and which should show which siblings of any animal under test are also under test. The highest ranked parents and their progeny would be added to the selected population of food-producing animals having the heritable capability of conservation.

3. The further experiment of isolating the progeny of sires A and B (see later) and measuring their conversion efficiency at a range of input levels has not yet been done.

4. For any species of food-producing animal, it may be useful to establish an upper limit for conservation of n-3 HUFA N a limit that if exceeded results in less efficient conservation. Such a limit may be amenable to genetic modification.

5. We expect that quantitative examination of relevant portions of the DNA or of the expressed mRNA or protein profiles of identified food-producing animals will lead to identification of the appropriate portions of the genome to be modified using known techniques for gene modification in order to bypass or accelerate the breeding programme and thereby produce animals that have superior performance (at least as indicated by their genomes) as sources of desirable n-3 HUFAs. Similarly, studies of portions of the DNA should reveal genetic markers for use as indicators of animals for which use of the test method in (1) above is warranted. See Example 3.

Method for Significance Testing for the 4100 Daughter, 11 Sire Dataset.

Significance testing was done with the help of the R statistical computing package; used for all analysis. Use of R is covered by the Gnu General Public License (GPL). The method for obtaining means and p-values for the 4100 dataset involved:

a) Mean fatty acid concentrations (percentages of total fatty acids) were calculated for each fatty acid for each sire within each herd.

b) Ranks were assigned to each sire (again, within each herd) based on their means.

c) Cumulative rank scores were obtained for each sire by adding up the rank scores the sire had been assigned across each herd. Possible cumulative rank scores ranged between 11 and 225, as all herds held daughters from all eleven sires represented except herds 4, 7, 11, 19 and 20 which had daughters from only 10, 10, 10, 9, and 10 sires respectively.

d) The significance of cumulative rank scores assigned was assessed via the following method;

1. daughters were permuted (i.e., scrambled) within herds by being assigned to a random sire, and the rank scoring procedure repeated for each sire within each herd. This was done 10000 times, so that a distribution of scores was obtained for each sire. Because the permutation breaks up the pedigree, these scores are posited to come from the null distribution (i.e., no heritable (or other factors other than chance could explain fatty acid concentrations in this distribution).

2. A p-value was then calculated for each sire based on these distributions by

2.1. summing the number of times that a permuted (scrambled) dataset produced a test statistic (sum of ranks) that was smaller than that observed in the real data (low rank sums indicating higher mean fatty acid levels) for each sire and then

2.2. dividing this sum by the number of times the datasets were permuted to get the p-value (10000 times here).

3. The p-values were then adjusted to take the number of sires (11) and fatty acids (2) being tested into account. The Bonferroni correction was used to control for multiple hypothesis testing. This involved dividing the standard significance level of alpha=0.05 by the number of hypotheses being tested (one for each sire, in each of C20.5 and C22.5).

### EXAMPLE 2

This Example describes a more general selection procedure. Referring to FIG. 2, the flowchart begins (box 201) with the acquisition of a population of food-producing animals to be tested, then provides a first stage (202) of the acquisition of sire and dam data about each one. “Dairy animals” are but one of many possible examples to which this flow chart and method can be applied. Optionally, only sire data is obtained; the dams may simply be uniquely identified although preferably the process is treated as the study of pedigrees for which ancestry of dams and sires is included. Identification may for example be by means of an ear tag or an implanted micro-chip for radio-frequency identification (RFID), or by DNA testing (as for prawns). A “first test procedure (including an augmented diet (205)) or a “second test procedure” ((204) normal diet without the supplement) is then selected (203).

The procedure includes the steps of (i) (first test procedure only) supplementing the diet of each identified animal to be tested with a supplement rich in at least one n-3 HUFA or a precursor thereof at an effective level (205) for a period of perhaps 3 to 7 days prior to collection of an appropriate sample including fats from each identified animal, (ii) collecting and analysing the fatty acid composition of the sample, then (iii) recording the percentage of total fatty acid comprised of at least one n-3 HUFA all in box 206. If the n-3 HUFA content is increased (207), the animal is rated accordingly (box 209). If one or both of the animal's parents are also highly rated, or provided consistently better rated progeny, or if siblings of the animal under test were highly rated, then the animal under test should be more highly rated (within decision box 208). If one or both parents are available for breeding use, they may be given a rating from this evidence. Example 1 illustrated the case of two half-brother sires producing a number of highly ranked progeny. They appear to carry a useful genotype and should be highly ranked, more so because having been selected for use as artificial breeding sires they would provide a “package” of desirable genes. The process should be carried out until a population of at least about 10-50 breeding animals is obtained. That population may be physically segregated or “informationally segregated” by which we mean that their identities and locations are known and access is possible. The process may be operated so that the only animals to be given a rating are sires or dams of the tested animals. This process can also be applied to food producing animals other than dairy cows. The procedure is repeated at each generation (box 209) in order to apply further selection pressure for the desired ability to conserve n-3 HUFAs, having due regard for retention of other known factors of genetic importance.

In a standard dairy herd context where commercial artificial insemination is used, a farm is offered semen from a small range of top quality bulls more or less at random, although the identity of each bull is known. Hence there will be relatively large groups of cows sharing the same sire. Groups also sharing the same dam will be smaller and usually of different ages.

Reference to repeating the test (in the lowest box) with progeny of identified animals is made in order to record those progeny having more heritable traits in succeeding generations. This flowchart includes an optional step of testing the animals for raised n-3 HUFA levels prior to supply of the enhanced diet.

It is known that too small an inbred population of organisms tends to develop “inbreeding depression” or reduction of fitness and so cannot be sustained. See D S Falconer “Quantitative Genetics” at p 248 (1964 reprint; Edinburgh: Oliver & Boyd). An effective number of less than about 10 breeding individuals of one generation is considered to be at particular risk although the actual population size is dependent on many specific factors such as sex ratios, existence of inter-generational breeding, and heritability and frequency of relevant genes; see Falconer (q.v.) at for example pp 50 and 68-74.

Therefore an effective self-sustaining population should include at least 10 breeding individuals of the same generation, and more preferably 50 or more. In order to provide a self-sustaining population having selected characteristics, it is desirable to repeat the selection process of Example 2 or other examples of this invention in order to create a sufficiently large breeding population.

### EXAMPLE 3

Genetic testing is a screening method; an alternative way to discover animals having an interesting genome. The tests are assumed to be correlated with phenotypic performance and avoid the time-consuming aspects of progeny testing. Any interesting genome is likely to be expressed as mRNA, or relevant proteins (or as greater quantities of relevant proteins), including enzymes, transport and binding proteins. This process may uncover a first genome adapted for maximising conservation of n-3 HUFAs at normal environmental levels, or a second genome (which might be substantially the same genome) that is more optimised for conserving higher levels of n-3 HUFAs if presented at higher than normal levels, or a third, anti-conserving genome having the function in nature of facilitating onwards conversion or metabolism of the n-3 HUFAs into other fatty acids and/or fatty acid by-products especially in situations with augmented intakes. “Conserving” in a pragmatic sense means that a maximised amount of dietary n-3 HUFAs or metabolites of their precursors can be recovered from food products. The above genomes may be functionally indistinguishable. They may occur separately in different animals. The genetic testing procedure may be carried out using DNA-related tests that search for previously established genetic markers (see below) within the test population.

Some relevant enzymes under control of an interesting genome having genes capable of expressing or controlling:

delta-6 desaturase, delta-5 desaturase, and fatty-acid chain elongation enzymes, absorbtion proteins, transport proteins and binding proteins acting on fatty acids, particularly any acting on n-3 HUFAs or their precursors as found within the gut lining, also plasma, cell membranes and other tissues (including mammary tissues).

Polymorphisms may be searched for. Multiple copies of a gene where expressed may affect the phenotype.

### Markers

Screening tests within a wide range of food-producing animals and may be extended to (for example) marine animals having life cycles very different from those of large populations of dairy cows under an artificial-insemination regime. Once recognisable (that is, distinctive) DNA sequences that have been shown in one species to serve as markers (being substantially co-inherited) for genes of direct relevance have been established, these markers may be employed when testing the same or other kinds of animal. Other forms of genetic indicator include testing for distinctive messenger RNA (m-RNA) markers; gene expression patterns to be expected when relevant genes are being expressed in active tissues. It will be necessary to identify novel marker transcripts which may occur together in recognisable sets. Yet another indicator is other expressed proteins which may be shown to be expressed in association with the desired genes. Such techniques for screening are well known to skilled workers in the art.

Further, once the actual genes involved have been established, it will be possible to create lines of transgenic animals having the desired capability for conserving n-3 HUFAs even under high dietary intakes, by means of insertion or deletion of particular genes within the genome of those animals, by genetic modification techniques well known in the appropriate arts. These techniques can bypass and can overcome limitations of conventional breeding. Another desired development in this area is the identification of genes involved in the conversion of n-3 HUFAs and the like into other compounds and the creation of artificial constructs for use in genetic modification that have the effect when expressed of modifying the rate of expression of relevant genes, by competition (having the same metabolic function as known enzymes), or by blocking the action of enzymes made by expression of those genes.

### EXAMPLE 4

### Other Food-Producing Animals

White meat from pigs and poultry is widely consumed around the world. Techniques for individual identification and for testing of progeny for ratio of various fatty acids while being fed on n-3 HUFA enriched diets (as previously described) are simple to implement for poultry and also for meat birds such as turkeys, ostriches and emus. The test protocols are as previously described. The samples to be tested may comprise eggs which can be repeatedly sampled without difficulty. Individual animal identification is also a relatively trivial matter. RFID transponders or eartags or the like may be particularly useful.

Marine or fresh-water animals that may be farmed have more diverse breeding methods and may present greater problems in terms of identification, control over breeding, and the like although the numbers of eggs or sperm released may be very large. These animals are more likely to be located at appropriate places in the food chain as consumers of enriched algal foods and many types are already well established as components of the human diet. It appears likely that DNA analysis will be an important tool for identification of marine animals and establishing their pedigree in all cases.

Molluscs: individuals may be of distinct sexes (abalones and Triton); united in Opisthobranchia and the Pulmonata (including Helix pomatia the edible land snail), while oysters for example are protandrous—the gonad produces sperms first then ova later. It appears likely that DNA analysis will be an important tool for identification of animals and establishing their pedigree in all cases.

Crustacea: Prawn breeding is already practised for example by the Commonwealth Scientific and Industrial Research Organisation (CSIRO) seeking improved growth rates in the Japanese prawn Penaus japonicus. Individuals are identified by DNA and management techniques include the accumulation of broodstock. Natural mating is used and the pedigree of high-performing individuals would be identified by DNA testing. In relation to the present invention, high-performing individuals may be identified by feeding high levels of n-3 HUFAs then testing sampled limbs. Alternatively, identification may be solely by identifying the relevant genes or associated markers or m-RNA.

Fishes: for example there is already a good deal of expertise in selecting salmon for growth rates, for creating genetically modified fish, “transgenic salmonids” and it would be feasible to apply the well-known techniques to select for n-3 HUFA conservation or similar properties on phenotypes and then, by using DNA analysis, select siblings of high-performing individuals for breeding. Alternatively, genetic analysis may be performed on samples taken from young fish and with knowledge of the genes that are involved, select particular individuals for breeding.

### INDUSTRIAL APPLICABILITY AND ADVANTAGES

The invention is intended to optimise the process within a food-producing animal of conserving n-3 HUFAs in food tissue that usually were originally supplied to the animal in its diet, so that the human diet is enhanced and health benefits ensue. Alternatively, the invention is intended to optimise the process of converting metabolic precursors, often of plant origin, into n-3 HUFAs that become incorporated into a food. The foods (such as meat, eggs, and dairy products) made from animals bred or made according to the invention will more economically provide n-3 HUFAs to the human diet as compared to products not from animals so selected. Subsequent food processing selective for particular fatty acids may be avoided.

The invention identifies those animals from within a population that are genetically best suited to ingest n-3 HUFAs or their metabolic precursors and to conserve and supply n-3 HUFAs for human consumption within a food product such as eggs, milk, meat or a processed extract.

Improvements in the health of individuals and a healthier population, in terms of cardiovascular disease in particular, (but not limited to that disease) is generally believed at the time of filing to result from a higher intake of the n-3 HUFAs, because very few persons normally ingest the recommended daily amount (such as 240 mg/day EPA & DHA) and modern food processing and storage tends to reduce the n-3 HUFAs. We show (as in Example 1) an about 30% increase in EPA from cows that are daughters of two related sires. Although that would correspond only to a small increase of about 7.5 mg/day of EPA in the daily intake of EPA in populations consuming high levels (up to about 30 grams a day) of butterfat, such an increase should lead to health effects which (whilst they may be difficult to measure directly at the individual level) should have a measurable effect when assessed on a population basis (taking due account of co-existing variables such as trends in diet of course).

By utilising a relatively greater ability of selected animals to convert an intake of n-3 into a palatable foodstuff with significant n-3 HUFA and by reducing the need for costly supplementation and where the invention is demonstrated to work on populations of food producing animals which have been provided with only a low of level n-3 supplementation in their diet economic production of high volume n-3 HUFA tissues will be enabled by a lowering of the threshold at which low level supplementation becomes effective. In addition, the need for expensive processing (such as microencapsulation) of n-3 HUFAs for addition to harvested and/or processed animal foods will be reduced. The production of such products could potentially over time facilitate a shift in the approach to marketing or regulating such products taking advantage of the potential benefits to individual as well as population health since it could make supplementation of a significant proportion of the whole human population economically feasible.

Likewise, exerting genetic selection pressure over entire populations of food producing animals should have a measurable effect at the population level, with no change to the dietary customs of the human population. A related advantage of the invention is that the higher intake of the n-3 HUFAs is achieved by people while eating and/or drinking normal diets (though from animals modified according to the invention) and without the disadvantages inherent in particular preparations of separately ingested dietary supplements (such as oxidation on storage, and ingestion of associated saturated fats).

The upper limit of increase in the n-3 HUFA content of foods derived from animals that have been selected according to the invention, when fed with a commercially feasible and acceptable diet including n-3 HUFAs of algal or plant origin, and/or with metabolic precursors of n-3 HUFAs, cannot easily be predicted. It would vary with the type (phylum, genus, and species) of animal and the form of the diet for example.

Finally, it will be understood that the scope of this invention as described and/or illustrated herein is not limited to the specified embodiments. Those of skill will appreciate that various modifications, additions, known equivalents, and substitutions are possible without departing from the scope and spirit of the invention as set forth in the following claims.

1. A food-producing animal selected from the range of food-producing animals as herein defined, characterised in that the animal has a heritable capability for conservation (as herein defined) of at least one compound selected from a range including: the n-3 HUFAs: EPA (eicosapentaenoic acid), DHA (docosahexanoic acid) and DPA (docosapentaenoic acid) and metabolic precursors thereof, so that the animal is capable of producing a food product including a higher level of at least one of the n-3 HUFAs as compared to levels of n-3 HUFAs in foods derived from animals lacking said heritable capability.

2. A food-producing animal selected from the range of food-producing animals as herein defined, characterised in that when fed with a diet including a supplementary amount of at least one compound selected from a range including: the n-3 HUFAs: EPA (eicosapentaenoic acid), DHA (docosahexanoic acid) and DPA (docosapentaenoic acid) and metabolic precursors thereof, the food-producing animal demonstrates a heritable capability of conserving at least one of the n-3 HUFAs provided within an augmented diet having the at least one compound at an effective level so that the animal is capable of producing a food product including a higher level of at least one of the n-3 HUFAs as compared to levels in foods derived from animals either lacking said heritable capability or not provided with the augmented diet.

3. A breeding population of food-producing animals comprised of food-producing animals as claimed in claim 1, characterised in that the population of food-producing animals exists, whether physically brought together or aggregated in an informational manner, in a sufficient number to be capable of transmitting the heritable capability to progeny of the population without adverse genotypes arising in the progeny owing to inbreeding, and wherein the number is at least in the range of from 10 to 50 breeding animals.

4. A method for obtaining members of a population of food-producing animals as claimed in claim 3 characterised in that the method involves the steps of:
a) obtaining access to a base population of individually identified food-producing animals, each animal having at least one identified parent,
b) carrying out a first test procedure on members of the base population; the first test procedure including the steps of (i) supplementing the diet of each animal to be tested with a supplement rich in at least one n-3 HUFA or a precursor thereof at an effective level for a period, (ii) collection of an appropriate sample from the animal, (iii) collecting and analysing the fatty acid composition of the sample, then (iv) recording the amount and percentage of total fatty acid comprised of at least one n-3 HUFA, and
c) identifying and if necessary separating out the animals providing the highest test results as comprising the selected population of animals.

5. A method as claimed in claim 4 for obtaining a selected population of food-producing mammals characterised in that the method involves the steps of:
a) obtaining access to a base population of individually identified animals, each animal having an identified sire selected from within a limited group of sires,
b) carrying out the first test procedure on members of the base population,
c) ascertaining the ranking of the sires of the members within the group of sires in terms of measurements derived from the first test procedure of samples taken from their progeny and ranking those sires whose progeny tends to return the highest amounts of n-3 HUFAs in the first test procedure relative to unrelated companions as the highest ranked sires, and
d) identifying and if necessary separating out the animals that are the progeny of the highest ranked sires as comprising the selected population of animals.

6. A method as claimed in claim 5 for obtaining a selected population of food-producing animals characterised in that the animals that are (a) the progeny of the highest ranked sires and (b) are themselves highly ranked in terms of n-3 HUFA measurements as determined by the first test procedure are defined as comprising the selected group of animals.

7. A method as claimed in claim 4 for obtaining a selected population of food-producing animals characterised in that the method uses a second test procedure that does not include the step in the first test procedure of supplementing the diet of each animal to be tested with a supplement rich in at least one n-3 HUFA or a precursor thereof at an effective level for a period before taking the sample.

8. A method as claimed in claim 5 for obtaining a selected population of food-producing animals characterised in that the method includes the following test procedure instead of the first or the second test procedures:
a) carrying out a third test procedure on members of the base population; the third test procedure including the steps of (i) collecting a sample of genetic material from the animal under test and analysing the genetic composition of the sample, then (ii) recording the genetic composition of the animal in terms of at least one of: genes relevant to conservation of the n-3 HUFAs, genetic markers associated with genes relevant to conservation of the n-3 HUFAs, and expressed m-RNA indicative of genes relevant to conservation of the n-3 HUFAs.

9. A method for maintaining a selected group of food-producing animals characterised in that the method includes the steps, aken for each generation, of
a) repeating the procedure of ascertaining the performance of the food-producing animals within the selected population in terms of n-3 HUFA measurements from their progeny according to the first test method,
b) ranking those food-producing animals whose progeny return the highest proportions of n-3 HUFA fats in the tests as the highest ranked breeding animals, and
c) breeding from the highest ranked breeding animals.

10. A method for maintaining a population of food-producing animals as claimed in claim 9, characterised in that the extent of inbreeding that may occur is minimised by the step of endeavouring to breed between animals that are as distantly related as possible, so that the population does not develop a high frequency of recessive genes and so that the effective breeding number of the population is maximised.

11. Reproductive material carrying genetic material capable of contributing to the genomes of a food-producing animal as claimed in claim 3 characterised in that the reproductive material has been selected from a range including spawn, milt, eggs and semen; embryos, larvae, stem cells, and intermediate and final products of genetic engineering including genetic constructs.

12. A foodstuff or manufactured foodstuff of animal origin, characterised in that the foodstuff is obtained from at least one animal obtained by a process as claimed in claim 4, and includes a raised proportion of at least one compound selected from the range including: the n-3 HUFAs: EPA (eicosapentaenoic acid), DHA (docosahexanoic acid) and DPA (docosapentaenoic acid) and metabolic precursors thereof.

13. A dairy foodstuff according to claim 12 characterised in that the foodstuff is derived from secreted milk from one or more dairy animals obtained by a process as claimed in claim 4, so that the dairy foodstuff has a raised proportion of n-3 HUFAs.

14. A foodstuff of avian origin according to claim 12 characterised in that the foodstuff comprises at least one egg or derivatives thereof.

15. A foodstuff according to claim 12 characterised in that the foodstuff comprises a body tissue selected from the range including meat or offal or blood.

16. A foodstuff according to claim 15 characterised in that the foodstuff comprises a at least one manufactured product made from a pooled product; the at least one product including without limitation meat or other tissue-based products such as sausage meat, mince, processed chicken, and other packages or compositions including parts from more than one individual animal.
